(19) United States
(12) Patent Application Publication
(10) Pub. No.: US 2009/0005413 A1
(43) Pub. Date: Jan. 1, 2009

(54) Novel Salt of Montelukast

(75) Inventors: Osman Achmatowicz, Warszawa (PL); Krzysztof Wisniewski, Pruszkow (PL); Jan Ramza, Warszawa (PL); Wieslaw Szelejewski, Warszawa (PL); Barbara Szechner, Warszawa (PL)

Correspondence Address: MATTHIAS SCHOLL, 14781 MEMORIAL DRIVE, SUITE 1319, HOUSTON, TX, 77079, US

(73) Assignees: INSTYTUT FARMACEUTYCZNY, WARSZAWA (PL); ZAKLADY FARMACEUTYCZNE POLPHARMA SA, STAROGARD GDANSKI (PL)

(21) Appl. No.: 11/577,721

(22) PCT Filed: Oct. 21, 2005
(86) PCT No.: PCT/PL2005/000067

§ 371 Date: Mar. 4, 2008

(30) Foreign Application Priority Data
Oct. 22, 2004 (PL) P-370850

(51) Int. Cl. A61K 31/47; C07D 215/18

(52) U.S. Cl. 514/311; 546/174

(57) ABSTRACT

The invention relates to a novel salt of montelukast with tert-butylamine and its use in the process for the preparation of highly pure free montelukast acid and/or pharmaceutically acceptable salts thereof, in particular montelukast sodium.

### CROSS-REFERENCE TO RELATED APPLICATIONS

This is a National Stage Application of International Patent Application No. PCT/PL 2005/000067, with an international filing date of Oct. 21, 2005, which is based on Polish Patent Application No. P-370850, filed Oct. 22, 2004. The contents of both of these specifications, including subsequent amendments thereto, are incorporated herein by reference.

### BACKGROUND OF THE INVENTION

1. Field of the Invention

This invention relates to a novel salt of (R,E)-(1-{1-{3-[2-(7-chloroquinolin-2-yl)ethenyl]-phenyl}-3-[2-(1-hydroxy-1-methylethyl)phenyl]-propylsulfanylmethyl}cyclopropyl)acetic acid. Furthermore, the invention relates to the process for the preparation of highly pure (R,E)-(1-{1-{3-[2-(7-chloroquinolin-2-yl)ethenyl]-phenyl}-3-[2-(1-hydroxy-1-methylethyl)phenyl]-propylsulfanylmethyl}cyclopropyl)acetic acid and/or its pharmaceutically acceptable salts.

2. Description of the Related Art

(R,E)-(1-{1-{3-[2-(7-chloroquinolin-2-yl)ethenyl]-phenyl}-3-[2-(1-hydroxy-1-methylethyl)phenyl]propyl-sulfanylmethyl}cyclopropyl)acetic acid (1),

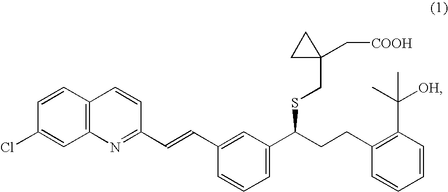

also known under its international non-proprietary name (INN) as montelukast, is a leukotriene D4 antagonist. Montelukast is indicated for the prophylaxis and chronic treatment of asthma in adult and pediatric patients. It is also indicated for the relief of symptoms of seasonal allergic rhinitis and for perennial allergic rhinitis in adult and pediatric patients. Montelukast sodium, the sodium salt of montelukast, is available in a number of oral formulations including tablets, chewable tablets, and oral granules. Two methods of synthesizing the sodium salt of (R,E)-(1-{1-{3-[2-(7-chloroquinolin-2-yl)ethenyl]phenyl}-3-[2-(1-hydroxy-1-methylethyl)phenyl]propylsulfanylmethyl}cyclopropyl)acetic acid (monteluksat sodium) are reported in the art.

In the first method, as reported in Patent Application Publication No. EP 0480717 A1, the synthesis of montelukast involves the coupling of methyl 1-(sulfanylmethylcyclopropyl)acetate cesium salt with 2-(2-(3(S)-(3-(2-(7-chloro-2-quinolinyl)-ethenyl)phenyl)-3-methanesulfonyloxypropyl)phenyl)-2-propanol, followed by hydrolysis of the obtained product to (R,E)-(1-{1-{3-[2-(7-chloroquinolin-2-yl)ethenyl]phenyl}-3-[2-(1-hydroxy-1-methylethyl)phenyl]propylsulfanylmethyl}-cyclopropyl)acetic acid, which is then converted directly to its sodium salt. In that process, chromatographic techniques are used for purification of the methyl ester intermediate, which limits the industrial usability of the method. In addition, montelukast sodium obtained by this method is an oily substance which may be converted to an amorphous form by lyophilization, a process not economically-viable on the industrial scale.

In the second method, as reported in the International Patent Application WO 9518107, another method of synthesizing montelukast sodium is disclosed. The method is based on King et al., J. Org. Chem., 1993, 58, 3731-3735, and comprises the use of dilithium dianion of (1-sulfanylmethylcyclopropyl)acetic acid as a nucleophilic reagent in substituting the methanesulfonyl moiety of 2-(2-(3S)-(3-(2-(7-chloro-2-quinolinyl)ethenyl)-phenyl)-3-hydroxypropyl)phenyl)-2-propanol mesylate. The thus obtained crude acid is isolated by acidifying the reaction mixture and converted to the dicyclohexylamine salt which is recrystallized and converted again into the free montelukast acid and further into its sodium salt. According to WO 9518107, recrystallization of montelukast sodium from the toluene/acetonitrile solution gives a crystalline form of montelukast sodium. However, the enclosed X-ray powder diffraction pattern of that product shows that, in fact, it is only a semi-crystalline substance. Specifically, the XRPD spectrum is characterized by poorly shaped diffraction peaks and the presence of the so called “amorphous halo”. Such a substance is not particularly suitable for formulating pharmaceutical dosage forms due to insufficient uniformity and lack of reproducibility of the product from batch to batch.

It is known to those skilled in the art that solubility profiles of amorphous solids may differ from those of crystalline ones. Therefore, the bioavailability of amorphous and crystalline solids is also different. Furthermore, other physico-chemical properties of amorphous solids may differ, as well, depending on the manufacturing conditions. For example, according to WO 03066598, amorphous montelukast sodium prepared by lyophilization is highly hydrated and very hygroscopic, whereas the substance prepared according to WO 03066598 is anhydrous. In WO 03066598 the process for preparation of anhydrous amorphous sodium montelukast is proposed, comprising precipitating montelukast sodium from its solution in a halogenated hydrocarbon C₁-C₂ or in an aromatic hydrocarbon C₇-C₈, with the use of an aliphatic hydrocarbon C₅-C₇ or a cyclic hydrocarbon C₅-C₈.

Thus, a vast divergence of views concerning defining and characterizing the polymorphic form of montelukast sodium that would be most suitable for pharmaceutical formulations is observed in the art. Difficulties in preparing a uniform crystalline form of montelukast sodium and the instability of its amorphous form make investigating new crystalline forms of montelukast having better stability and uniformity desirable.

Furthermore, there still exists a need to simplify the manufacturing process for preparation of montelukast acid and its salts and to rationalize associated manufacturing costs. Preparing free montelukast acid of greater degree of chemical purity is of special interest.

Chemical purity of montelukast acid prepared according to a process described in WO 9518107 depends, among other factors, on the purity of the methanesulfonate salt used as a starting material, as well as on conditions of the reaction of methanesulfonyl chloride with the diol. Analysis of the reaction mechanism indicates that increased reaction temperature results in a decreased selectivity of mesylation of the secondary hydroxyl group. The type of the reaction solvent used has an impact on reactivity of the diol. An intramolecular substitution, resulting in formation of a cyclic ether, is observed in acidic medium (i.e. in the presence of diisopropylethylamine hydrochloride) at a temperature above −10° C.

It is the aim of this invention to develop an improved process for preparation of montelukast that would provide a highly pure substance possessing advantageous physico-chemical properties, high degree of crystallinity and thermodynamic stability, and therefore having appropriate processing parameters facilitating its formulating into the pharmaceutical dosage forms.

The aim of the invention has been achieved by obtaining a novel salt of (R,E)-(1-{1-{3-[2-(7-chloroquinolin-2-yl)ethenyl]phenyl}-3-[2-(1-hydroxy-1-methyl-ethyl)phenyl]propylsulfanylmethyl}cyclopropyl)acetic acid.

### BRIEF SUMMARY OF THE INVENTION

The invention provides in one aspect a novel tert-butylamine salt of (R,E)-(1-{1-{3-[2-(7-chloroquinolin-2-yl)ethenyl]-phenyl}-3-[2-(1-hydroxy-1-methylethyl)phenyl]-propylsulfanylmethyl}cyclopropyl)acetic acid. The tert-butylamine salt of montelukast is readily isolable in a substantially crystalline form and may be used as a means for the purification of free montelukast acid.

In another aspect, the invention provides the use of the novel tert-butylamine salt of montelukast in preparing highly pure (R,E)-(1-{1-{3-[2-(7-chloroquinolin-2-yl)ethenyl]-phenyl}-3-[2-(1-hydroxy-1-methylethyl)phenyl]-propylsulfanylmethyl}cyclopropyl)acetic acid and/or its pharmaceutically acceptable salts.

In a preferred embodiment of that aspect of the invention, it is provided the use of the new tert-butylamine salt of montelukast in preparing montelukast sodium.

Furthermore, the invention provides pharmaceutical compositions comprising the novel tert-butylamine salt of montelukast together with pharmaceutically acceptable carriers and/or excipients.

### BRIEF DESCRIPTION OF THE DRAWINGS

The objects and advantages of the present invention will become more readily apparent after reading the ensuing description of the non-limiting illustrative embodiment and viewing the accompanying drawings, in which

FIG. 1 shows X-ray powder diffraction pattern of the tert-butylamine salt of (R,E)-(1-{1-{3-[2-(7-chloroquinolin-2-yl)ethenyl]phenyl}-3-[2-(1-hydroxy-1-methylethyl)phenyl]-propylsulfanylmethyl}cyclopropyl)acetic acid (“tert-butylamine salt of montelukast”); and

FIG. 2 shows X-ray powder diffraction pattern of (R,E)-(1-{1-{3-[2-(7-chloroquinolin-2-yl)ethenyl]phenyl}-3-[2-(1-hydroxy-1-methylethyl)phenyl]-propylsulfanylmethyl}cyclopropyl)acetic acid (“montelukast acid”).

### DETAILED DESCRIPTION OF THE INVENTION

The novel tert-butylamine salt of montelukast can be easily isolated from the reaction mixture in the crystalline form, and then, if necessary, purified by recrystallization from typical organic solvents to reduce impurities down to a pharmaceutically-acceptable level. The tert-butylamine salt of montelukast can be easily converted to the free (R,E)-(1-{1-{3-[2-(7-chloroquinolin-2-yl)ethenyl]phenyl}-3-[2-(1-hydroxy-1-methylethyl)phenyl]propyl-sulfanylmethyl}-cyclopropyl)acetic acid, that, if necessary, is further converted into another pharmaceutically acceptable salt, for example, into the sodium salt.

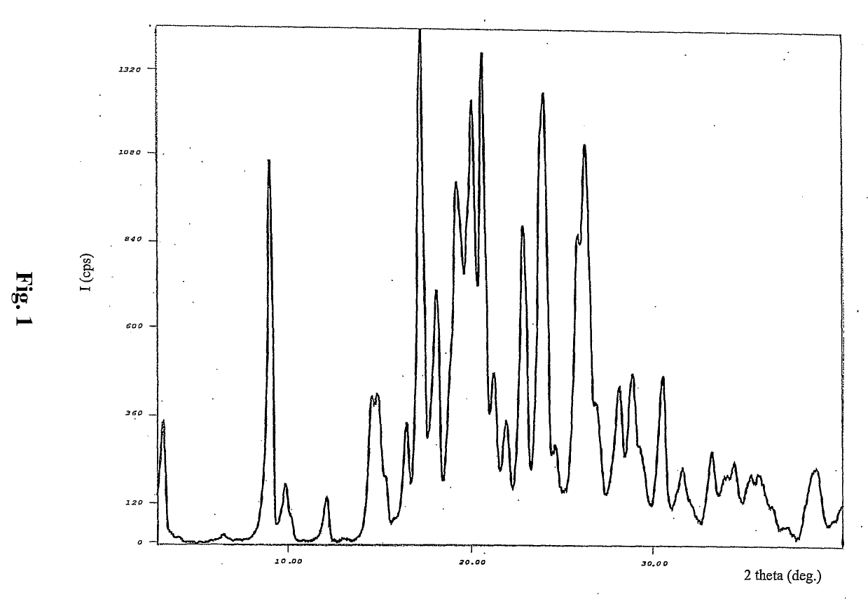

The tert-butylamine salt of montelukast is characterized by an X-ray powder diffraction pattern substantially presented in Table 1 and in FIG. 1.

TABLE 1
XRPD data of the tert-butylamine salt of montelukast.

| d (Å) | 2θ (°) | I/I₀ (%) |
|---|---|---|
| 3.30 | 26.74 | 24 |
| 8.89 | 9.94 | 75 |
| 9.67 | 9.13 | 12 |
| 11.92 | 7.42 | 9 |
| 14.26 | 6.21 | 28 |
| 14.67 | 6.03 | 29 |
| 15.10 | 5.86 | 13 |
| 16.22 | 5.46 | 24 |
| 17.03 | 5.20 | 100 |
| 17.86 | 4.96 | 49 |
| 19.87 | 4.46 | 87 |
| 20.43 | 4.34 | 96 |
| 21.01 | 4.22 | 33 |
| 21.69 | 4.09 | 24 |
| 22.67 | 3.92 | 62 |
| 23.86 | 3.73 | 85 |
| 24.41 | 3.64 | 19 |
| 25.61 | 3.48 | 59 |
| 26.15 | 3.40 | 77 |
| 26.74 | 3.33 | 27 |
| 27.96 | 3.19 | 31 |
| 28.69 | 3.11 | 33 |
| 30.35 | 2.94 | 33 |
| 31.43 | 2.84 | 15 |
| 33.01 | 2.71 | 18 |
| 34.24 | 2.62 | 16 |
| 35.13 | 2.55 | 14 |

DSC diagram of the tert-butylamine salt of montelukast recrystallized from toluene shows the melting point, determined as “the onset peak”, equal to 128.09° C.

The tert-butylamine salt of montelukast has advantageous physico-chemical properties, is non-toxic and well-soluble in typical solvents, for example, in lower alcohols and in acetone. Hence, it can be used as pharmaceutically-acceptable salt in manufacturing the pharmaceutical compositions for the treatment of conditions mediated by leukotrienes, such as asthma, inflammations and allergies. Handbook of Pharmaceutical Salts, ed. P. H. Stahl. C. G. Wermuth, Verlag Helvetica Chimica Acta, 2002.

For therapeutic applications, the tert-butylamine salt of montelukast is formulated into the pharmaceutical compositions comprising therapeutically effective amount of the salt together with at least one or more pharmaceutically-acceptable carrier and/or diluent.

The pharmaceutical composition according to the invention is administered to a patient in a need of such a treatment in a suitable pharmaceutical dosage form, by a route appropriate for that dosage form, for example, orally, parenterally (e.g., intravenously, intramuscularly, subcutaneously), pulmonarily, or intranasally.

Choice of dose of the tert-butylamine salt of montelukast and the dosage regimen depends on the type of disease, age, weight and condition of the patient, and they are determined by those skilled in the art on the basis of known procedures of treatment and prevention of such diseases. Preferred dose of the salt according to the invention is 5-10 mg per day for adults and 2-5 mg per day for children, calculating on the basis of free montelukast acid. The daily dose is administered to the patient once per day or several times per day, separately or in a combination with other pharmacologically-active substances. The constituents of such combinations are administered concurrently, in the form of a single formulation, or as individual formulations. Alternatively, the formulations are administered subsequently, in the order and time intervals determined by those skilled in the art.

The pharmaceutical composition according to the invention are formulated in various dosage forms, well known to those skilled in the art, and described, e.g. in Remington's Pharmaceutical Sciences, XVI^{th} ed., Mack Publ. Co., 1980.

The pharmaceutical formulations for oral administration comprise tablets, coated tablets, powders, granules, pellets or capsules comprising solid pharmaceutically acceptable carriers such as corn starch, lactose, sucrose, sorbitol, talc, mannitol or dicalcium phosphate. The tablets or granules are coated or otherwise processed to obtain a unit dosage form providing advantageous prolonged activity, if needed. A number of various substances are used for preparing such coating layers, comprising polymeric acids and the mixtures thereof with such substances as shellac, cetyl alcohol or cellulose acetate.

One could also consider administering pharmaceutical compositions comprising the tert-butylamine salt of montelukast in the form of preparations for injection or infusion. Such formulations comprise sterile aqueous, aqueous-organic and non-aqueous solutions, suspensions, dry powders, and tablets for preparing solutions or for implantation. Excipients that ensure uniform distribution of the active ingredient in the liquid phase, used for preparing suspensions comprise polysorbates, lecithin, polyoxyethylene/polyoxypropylene copolymers, peptizing agents such as, e.g., phosphates, polyphosphates and citrates, water-soluble polymers such as, e.g., carboxymethylcellulose, methylcellulose, polyvinylpyrrolidone, resins or gelatin. The injectable compositions can contain pharmaceutically-acceptable excipients such as, e.g., pH-adjusting agents and buffers, tonicity modifiers and preservatives. The dry powders are designated for preparing solutions or suspensions ex tempore, by diluting them with appropriate solvents.

The pharmaceutical compositions according to the invention are also in the form suitable for pulmonary (nasal or buccal inhalation) or intranasal administration. Inhalation formulations comprise such forms like aerosol spray from pressurized packs or nebulizers, or dry powders. Aerosols, which are formulated as a suspension or solution of the active ingredient in suitable propellants, such as fluorocarbons or hydrocarbons, may be inhaled with the aid of a metered dose inhalation aerosol. Intranasal drops or aerosol sprays contain the active ingredient dissolved or suspended in a carrier, e.g., a vegetable oil, low-molecular weight polyethylene glycol, glycerol, sorbitol, triglycerides of fatty acids. Further constituents of nasal formulations are the tonicity modifiers and buffering agents that provide appropriate osmolarity (within the range 270-330 mOsm) and pH (within the range 4.0-7.0) of the composition. The appropriate auxiliary agents comprise sodium chloride, glucose, mannitol, lactose, kollidon, and phosphate buffer. Furthermore, the formulation can contain surfactants acting as solubilizers, emulsifiers and surface-tension reducing agents, such as, e.g., sorbitan esters, viscosity enhancers, such as, e.g., methylcellulose, antioxidants, such as, e.g., sodium edetate, sodium pyrosulfite, sodium ascorbate or palmityl ascorbate and preservatives, such as, e.g., benzalkonium chloride, phenylmercury borate or nitrate, chlorobutanol, methyl hydroxybenzoate, bronopol, benzyl alcohol, butylhydroxytoluene (BHT) and butylhydroxyanisole (BHA).

The invention also provides a process for the preparation of highly pure (R,E)-(1-{1-{3-[2-(7-chloroquinolin-2-yl)ethenyl]phenyl}-3-[2-(1-hydroxy-1-methylethyl)phenyl]propylsulfanylmethyl}cyclopropyl)acetic acid (1)

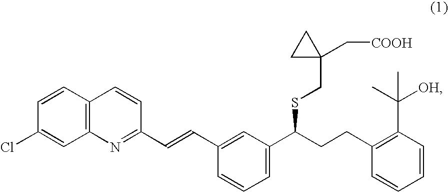

and/or pharmaceutically acceptable salts thereof, comprising the steps of:
(a) reacting the sulfonate derivative of (S)-1-{3-[2-(7-chloroquinolin-2-yl)ethylene]phenyl}-3-[2-(1-hydroxy-1-methylethyl)phenyl]propan-1-ol of the formula (2),

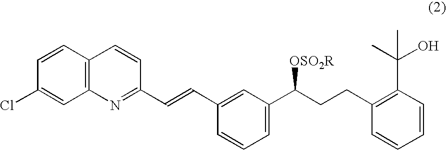

with a dianion of 1-(mercaptomethyl)-cyclopropaneacetic acid of the formula (3)

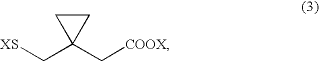

wherein
R represents an alkyl or aryl moiety, and
X represents a counterion, such as sodium;
(b) reacting the resulting crude (R,E)-(1-{1-{3-[2-(7-chloroquinolin-2-yl)ethenyl]phenyl}-3-[2-(1-hydroxy-1-methylethyl)phenyl]propylsulfanylmethyl}cyclopropyl)acetic acid with tert-butylamine to obtain the tert-butylamine salt of montelukast;
(c) isolating the tert-butylamine salt of montelukast from the reaction mixture;
(d) optionally, recrystallizing the tert-butylamine salt of montelukast from a solvent;
(e) converting the tert-butylamine salt of montelukast to a free highly pure (R,E)-(1-{1-{3-[2-(7-chloroquinolin-2-yl)ethenyl]-phenyl}-3-[2-(1-hydroxy-1-methylethyl)phenyl]-propylsulfanyl-methyl}cyclopropyl)acetic acid; and
(f) if required, converting the free acid from step (e) to a pharmaceutically-acceptable salt of montelukast.

The pharmaceutically acceptable salts that are obtained by the method according to the invention comprise salts of alkali metals and alkaline earth metals such as lithium, sodium, potassium, magnesium, calcium, ammonium salts, salts of amino acids, such as, e.g., L-ornitine and salts of organic amines such as, e.g., benzylamine, α-methylbenzylamine, N-methylbenzylamine; N,N-dimethylbenzylamine, phenethylamine, tribenzylamine, cyclopropylamine, cyclobutylamine, cyclopentylamine, cycloheksylamine, cycloheptylamine; N,N-dimethylcyclohexylamine, pyrrolidine N-methylpyrrolidine, piperidine, N-methylpiperidine, morpholine and other.

In particular, the process according to the invention is used for manufacturing alkali metals salts of montelukast, in particular montelukast sodium, in the following manner.

The starting material is alkyl- or arylsulfonate, preferably methanesulfonate of (S)-1-{3-[2-(7-chloroquinolin-2-yl)ethylene]phenyl}-3-[2-(1-hydroxy-1-methylethyl)phenyl]-propan-1-ol that is prepared by a method known per se, in a reaction of optically pure (S)-1-{3-[2-(7-chloroquinolino-2-yl)ethylene]phenyl}-3-[2-(1-hydroxy-1-methylethyl)phenyl]-propan-1-ol (ee>99.8%) with an appropriate sulfonyl chloride, e.g., with methanesulfonyl chloride, in the presence of diisopropylethylamine at a temperature below −10° C.

In the preferred embodiment of the invention, the dianion of (1-mercaptomethyl-cyclopropyl)acetic acid of the formula (3) is generated from disodium salt of (1-mercaptomethyl-cyclopropyl)acetic acid, which salt is much easier to obtain then the dilithium one, provided that appropriate reaction parameters are maintained.

The disodium salt of (1-mercaptomethylcyclopropyl)acetic acid is prepared in the reaction of the acid with sodium alkoxide, e.g., sodium tert-butoxide or sodium sec-amylate, in a solution of aprotic dipolar solvent such as dimethylformamide (DMF), dimethylsulfoxide (DMSO), dimethylacetamide (DMA) or 1-methylpyrrolidone (NMP) at a temperature not exceeding 25° C. Next, a solution of the methanesulfonate is added to a solution of (1-sulfanylmethylcyclopropyl)acetic acid disodium salt in the same solvent. The reaction mixture is stirred at the temperature of the range from 0° C. to 30° C., preferably at about 15° C. After complete conversion of the reagents, the reaction mixture is diluted with an inert organic solvent and neutralized with aqueous solution of sodium chloride. Next, the crude acid is isolated from the organic phase by evaporating the solvent. The crude oily acid residue that can contain a number of impurities, such as a cyclic ether being a product of intramolecular substitution of the diol during its mesylation under acidic conditions, a product of methanesulfonic acid elimination, a product resulting from cis-isomerization reaction or a product resulting from a di-substitution reaction, is re-dissolved in an inert organic solvent and then treated with tert-butylamine. Toward this end, an equimolar amount of tert-butylamine is added to a solution of the acid at temperature about 40° C. monitoring precipitation of the solid salt from the cooled reaction mixture seeded with crystals of the product. The thus obtained salt of chromatographic purity exceeding 90% could be further purified by a single or multiple recrystallizations either from the same or from another solvent. In the preferred embodiment of the invention, the salt is recrystallized from an organic solvent such as toluene, ethyl acetate, acetone, methyl isobutyl ketone or from the mixtures thereof with other solvents such as, e.g., hexane, heptane, acetonitrile, diethyl ether or tert-butyl-methyl ether. More preferably, the salt is recrystallized from a solvent selected from the group comprising toluene/hexane, toluene/heptane, toluene/diethyl ether, ethyl acetate/hexane or acetone/hexane mixtures.

The chemical purity of the salt after recrystallization, as determined by HPLC, is greater than 98.0%, and preferably is greater than 99.0%.

The tert-butylamine salt of montelukast can be used as a pharmaceutically active ingredient as such, or it may be further converted into a different pharmaceutically-acceptable salt.

Towards this end, free montelukast acid is liberated from its tert-butylamine salt by treating the salt with an aqueous solution of an organic mono- or dicarboxylic acid or a buffer solution. Highly pure montelukast acid can be obtained after a work-up, involving isolation of the crude acid from an organic phase and, if required, additional recrystallization from methanol or ethanol. The chemical purity of the thus obtained montelukast acid, determined by HPLC, is greater than 99.0%, and preferably greater than 99.5%.

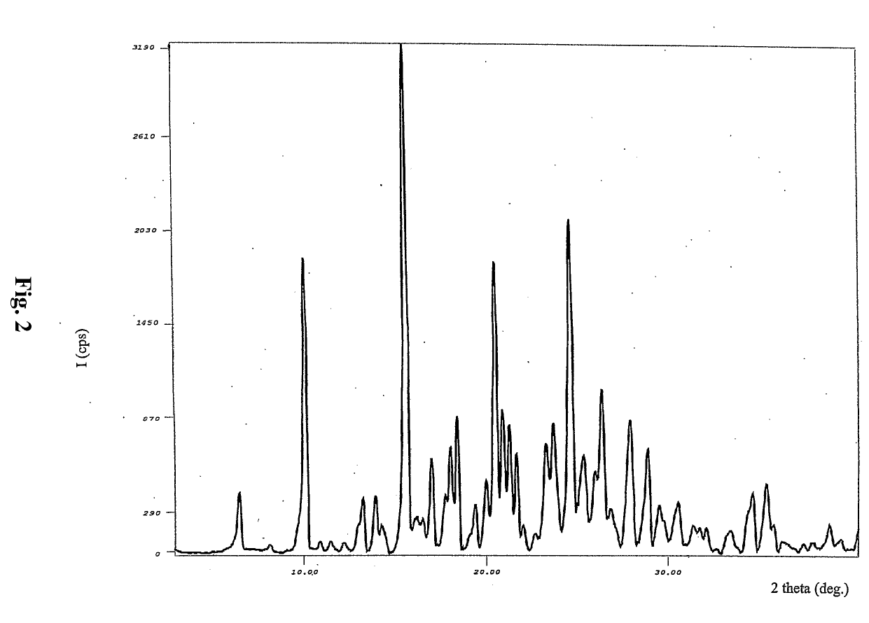

Montelukast acid, prepared according to a method of the invention, is characterized by an X-ray powder diffraction pattern substantially similar to that presented in Table 2 and in FIG. 2.

TABLE 2
XRPD data of montelukast acid

| d (Å) | 2θ (°) | I/I₀ (%) |
|---|---|---|
| 6.36 | 13.89 | 12 |
| 8.01 | 11.02 | 2 |
| 9.92 | 8.91 | 58 |
| 10.71 | 8.26 | 2 |
| 11.26 | 7.85 | 2 |
| 11.99 | 7.38 | 2 |
| 12.73 | 6.95 | 5 |
| 13.03 | 6.79 | 11 |
| 13.70 | 6.46 | 12 |
| 14.04 | 6.30 | 6 |
| 15.40 | 5.75 | 100 |
| 16.81 | 5.27 | 19 |
| 17.51 | 5.06 | 11 |
| 17.84 | 4.97 | 21 |
| 18.22 | 4.87 | 27 |
| 19.21 | 4.62 | 10 |
| 19.80 | 4.48 | 15 |
| 20.32 | 4.37 | 57 |
| 20.72 | 4.28 | 28 |
| 21.11 | 4.20 | 25 |
| 21.50 | 4.13 | 20 |
| 23.13 | 3.84 | 22 |
| 24.51 | 3.63 | 65 |
| 25.26 | 3.52 | 19 |
| 26.24 | 3.39 | 32 |
| 27.81 | 3.21 | 26 |
| 30.42 | 2.94 | 10 |
| 34.40 | 2.61 | 12 |
| 35.11 | 2.55 | 14 |

As illustrated in Table 3, the DSC diagram of montelukast acid prepared according to a method of the invention shows the following melting points, as determined by two methods (peak and onset).

TABLE 3
DSC of montelukast acid

| | |
|---|---|
| Melting point, ° C. | 155.61 |
| (acc. to peak extrapolation) | |
| Melting point, ° C. | 153.20 |
| (acc. to _{”}onset”) | |
| Enthalpy of fusion, J/g | −108.66 |

Free montelukast acid can be further converted to the sodium salt, by treatment with a source of sodium ions, for example sodium hydroxide, which is used in equimolar amount with the free acid.

The invention provides a novel salt of montelukast with tert-butylamine characterized by advantageous physico-chemical and pharmacological properties that constitute an active ingredient of the compositions useful for the treatment of asthma, inflammatory and allergic conditions. The novel salt of montelukast with tert-butylamine allows for an easy purification of the free montelukast acid and/or its conversion to the other pharmaceutically acceptable salts of high degree of chemical purity.

The following examples are provided to illustrate the invention. The examples are not meant to limit the scope of the invention as defined in the claims.

### EXAMPLES

The tert-butylamine salt of montelukast, as well as montelukast acid, has been characterized using the following methodologies.

a) X-Ray Powder Diffraction (XRPD)

XRPD patterns were collected for the radiation of CuKα at the wavelength λ=1.54056 Å as a function of relative intensity of diffraction peaks CuKα, the diffraction angle θ and intraplanar distances d. Patterns were collected with a Rigaku MINI FLEX diffractometer using the following parameters: 2θ range=3-40°, scanning rate=0.5 deg/min and step size=0.03 deg.

b) Thermal Analysis

Differential Scanning Calorimetry (DSC) was carried out with Mettler Toledo DSC 822 apparatus in a standard sealed aluminum pan, within the temperature range 40-200° C. Heating sequence consisted of a dynamic segment at the heating rate 10° C./min, preceded by an isothermic segment (40° C. for 3 minutes). The melting point and enthalpy of fusion were determined from the melt endotherm in the final heating scan. The melting point was determined by two methods: as “an extrapolated peak”, i.e. as an intersection point of tangents to the peak, and an “an onset”, i.e. the intersection point of a tangent to the baseline and a tangent to the increasing peak.

### EXAMPLE 1

### 2-(2-(3S)-(3-(2-(7-Chloro-2-quinolinyl)-ethenyl)phenyl)-3-hydroxypropyl)phenyl)-2-propanol methanesulfonate

2-(2-(3S)-(3-(2-(7-Chloro-2-quinolinyl)-ethenyl)phenyl)-3-hydroxy-propyl)phenyl)-2-propanol (70.0 g), DMF (175 mL) and diisopropylamine (35 mL) were placed under argon in a 1,000 mL flask, provided with a magnetic stirrer, thermometer, dropping funnel and a gas inlet. The mixture was cooled down to −15° C. in a dry ice/isopropanol bath and 14 mL of methanesulfonyl chloride was added dropwise within 1 hour, maintaining temperature below −15° C. The uniform reaction mixture was stirred for another hour and then 690 mL of cooled acetonitrile was added at −15° C. A precipitate has started to form after 1.5 hour of stirring at −20° C. The precipitate was filtered off under argon, washed with cold acetonitrile (400 mL), and then with cold hexane (200 mL). The product was dried under a stream of argon and then under reduced pressure (25 mbar) at room temperature to afford 78.3 g of loose, light yellow solid.

¹H NMR (CDCl₃): 8.11 (2H, m); 7.69 (5H, m); 7.41 (5H, m); 7.19 (3H, m); 5.70 (1H, dd); 3.25 (1H, m); 3.04 (1H, m); 2.76 (3H, s); 2.45 (1H, m), 1.92 (1H, s); 1.65 (6H, s).

### EXAMPLE 2

### tert-Butylammonium (R,E)-(1-{1-{3-[2-(7-chloroquinolin-2-yl)ethenyl]phenyl}-3-[2-(1-hydroxy-1-methylethyl)phenyl]-propylsulfanylmethyl}cyclopropyl)acetate (also referred to as the “tert-butylamine salt of (R,E)-(1-{1-{3-[2-(7-Chloroquinolin-2-yl)ethenyl]phenyl}-3-[2-(1-hydroxy-1-methylethyl)phenyl]propylsulfanylmethyl}cyclopropyl)acetic acid”, “tert-butylamine salt of montelukast”, and “montelukast tert-butylammonium”)

1-Methylpyrrolid-2-one (NMP) (200 mL) was placed in a 1,000 mL three-neck flask, provided with a mechanical stirrer, thermometer and gas inlet. 10.70 g of sodium t-butoxide was added with continuous stirring. A clear, violet solution was formed. Under vigorous stirring 1-(mercaptomethyl)-cyclopropaneacetic acid (8.09 g; 0.055 mmol) was added to this solution at 20° C. and temperature of the reaction mixture has risen to approximately 32° C. A pink suspension that has been formed was cooled down to 10° C. and the whole mixture was stirred for next 30° C. Within 2 minutes crystalline 2-(2-(3S)-(3-(2-(7-chloro-2-quinolinyl)-ethenyl)phenyl)-3-hydroxypropyl)phenyl)-2-propanol mesylate (30.0 g; 0.056 mmol) was added to the reaction mixture. The mixture was kept at 10° C. for 23 hours and then the reaction was quenched by pouring 500 mL of toluene cooled down to 5° C. to the homogenous, yellow reaction mixture. The resulting solution was poured into 500 mL of 10% brine, cooled down to 5° C. The whole mixture was stirred for 10 minutes and then left to allow for separation of layers. The product-containing organic layer was washed with 0.5 M solution of tartaric acid (250 mL) and two portions of water (2×250 mL). The solution of the product was transferred to a 1,000 mL round-bottom flask and toluene was evaporated under reduced pressure to afford approximately 28.0 g of very dense oil. The residue after evaporation was dissolved in 250 mL of fresh toluene and then tert-butylamine (15 mL) and 1.8 g of charcoal were added to the solution. The whole mixture was stirred at approximately 22° C. for 1 hour. The mixture was filtered under reduced pressure through a layer of Celite (15 g), the solids were washed twice with toluene (2×25 mL), and the obtained clear solution was transferred to a 1,000 mL three-neck flask provided with a mechanical stirrer, a thermometer and an argon inlet. The solution was seeded with 150 mg of the crystalline tert-butylamine salt of montelukast, recrystallized previously from toluene. The whole mixture was stirred at room temperature (20-25° C.). A white suspension was formed after approximately 16 hours that was stirred for next 32 hours. The suspension was filtered under reduced pressure of argon and the filter cake was washed with 200 mL of acetone. The product was dried at 22° C. under reduced pressure of argon (25 mbar) for 18 hours to yield 20.9 g (57%) of the tert-butylamine salt of montelukast as a white solid. Purity: 95.97% (by HPLC). The mother liquor was concentrated in vacuo to provide additional 7.3 g of the salt in the form of viscous oil. The combined precipitates of the tert-butylamine salt of montelukast, 30.45 g (purity 94.99% (HPLC)) and 23.66 g (purity 95.62% (HPLC)), were treated with 500 mL of toluene. The resulting dense suspension was vigorously stirred at 40° C. using a mechanical stirred for 16 hours and then at 20° C. for 3 hours. After filtering, washing with acetone (400 mL) and drying, 41.30 g of the title compound were obtained (76% yield). Purity: 98.27% (HPLC).

¹H NMR (500 MHz; CHCl₃): δ 8.09 (d, 1H, J=8.6 Hz), 8.05 (d, 1H, J=2.1 Hz), 7.72-7.63 (m, 4H), 7.47-7.06 (m, 9H), 3.99 (t, 1H, J=7.3 Hz), 3.93 (broad s, active H), 3.22-3.14 (m, 1H), 3.91-2.84 (m, 1H), 2.57 (AB, 2H, J=13.0 Hz), 2.37 (AB, 2H, J=15.7 Hz), 2.29-2.12 (m, 2H), 1.61, 1.59 (2×s, 6H), 1.22 (s, 9H), 0.56-0.34 (m, 4H).

¹³C NMR (125 MHz; CHCl₃): δ 177.0, 157.0, 148.4, 145.3, 143.8, 140.3, 136.5, 136.3, 135.7, 135.4, 133.5, 131.5, 129.0, 128.7, 128.6, 128.5, 127.9, 127.2, 127.1, 126.6, 126.2, 125.7, 125.6, 125.4, 122.3, 119.4, 73.7, 50.3, 49.1, 47.0, 42.0, 40.0, 39.5, 32.3, 31.9, 30.4, 17.2, 12.7, 12.3.

Combustion analysis: for C₃₉H₄₇ClN₂O₃S calcd.: C, 71.05%; H, 7.19%; N, 4.25%; S, 4.86%; found. C, 70.94%; H, 7.23%; N, 4.28%; S, 4.89%.

HRMS: for C₃₅H₃₇ClNO₃S calculated 586.2177, found 586.2201 ([M+H]⁺).

### EXAMPLE 3

### (R,E)-(1-{1-{3-[2-(7-Chloroquinolin-2-yl)ethenyl]phenyl}-3-[2-(1-hydroxy-1-methylethyl)phenyl]-propylsulfanylmethyl}cyclopropyl)acetic acid

The tert-butylamine salt of montelukast obtained as in Example 2 (76.11 g, purity 98.42% (HPLC)) and 800 mL of toluene were placed in a 4,000 mL separatory funnel, provided with a mechanical stirrer and powder- and argon inlets, washing the powder inlet with additional 200 mL of toluene. Under vigorous stirring, 0.554 M solution (150 mL) of tartaric acid was added to the suspension. The stirrer was turned off after 20 minutes and the mixture was left until layers have separated. 950 mL of water was added to the clear organic layer in the funnel and the mixture was stirred vigorously for 10 minutes. THF (250 mL) was added to the resulting dense emulsion. After 2.5 hours, partially separated water (approx. 400 mL) was removed. Next, a solution of sodium hydroxide in methanol (215 mL; 0.521 M) was added to the remaining emulsion. After 2 min. of stirring, the stirrer was turned off and two clear layers were formed. The toluene layer was filtered through a 0.45 μm nylon filter and reduced in volume to 250 mL. The yellow, aqueous layer was acidified with 0.554 M solution of tartaric acid to pH 4, and the separated oil was extracted with one 400 mL portion of chloroform. The chloroform extract was filtered through a 0.45 μm nylon filter and the solvents were evaporated in vacuo. The residue was treated with 250 mL of methanol and dissolved by heating to 40° C. The solution was left at room temperature for crystallization. The resulting light yellow precipitate was filtered, washed with 200 mL of methanol and dried under reduced pressure to afford 50.5 g (75% yield) of free (R,E)-(1-{1-{3-[2-(7-chloroquinolin-2-yl)ethenyl]phenyl}-3-[2-(1-hydroxy-1-methylethyl)phenyl]-propylsulfanylmethyl}cyclopropyl)acetic acid. Purity: 99.50% (HPLC).

¹H NMR (500 MHz; CHCl₃): δ 8.10 (d, 1H, J=8.6 Hz), 8.05 (d, 1H, J=2.1 Hz), 7.77-7.50 (m, 4H), 7.47-7.08 (m, 9H), 4.01 (t, 1H, J=7.2 Hz), 3.22-3.14 (m, 1H), 3.96-2.89 (m, 1H), 2.53 (AB, 2H, J=13.1 Hz), 2.46 (AB, 2H, J=16.2 Hz), 2.26-2.13 (m, 2H), 1.62, 1.61 (2×s, 6H), 0.54-0.42 (m, 4H).

¹³C NMR (125 MHz; CHCl₃): δ 175.3, 157.0, 148.1, 145.2, 143.5, 140.2, 136.5, 136.4, 135.8, 135.5, 131.5, 129.0, 128.7, 128.6, 128.5, 127.6, 127.3, 127.2, 126.6, 126.4, 125.7, 125.6, 125.4, 119.1, 73.9, 50.3, 40.2, 39.9, 38.7, 32.2, 31.8, 31.7, 16.6, 12.5, 12.3.

Combustion analysis: for C₃₅H₃₆ClNO₃S calculated C, 71.71%; H, 6.19%; N, 2.39%; S, 5.47%; found. C, 72.00%; H, 6.14%; N, 2.33%; S, 5.58%.

### EXAMPLE 4

### (R,E)-(1-{1-{3-[2-(7-Chloroquinolin-2-yl)ethenyl]phenyl}-3-[2-(1-hydroxy-1-methylethyl)phenyl]-propylsulfanylmethyl}cyclopropyl)acetic acid

The acid obtained in Example 3 (47.50 g; purity 99.50%) was combined with 7.38 g of the acid from another batch (purity 98.1%) (in total: 54.88 g). The combined batches were placed in a 1,000 ml one-neck flask, provided with a magnetic stirrer and an argon inlet and 500 mL of toluene was added. To the stirred suspension 0.521 M solution of sodium hydroxide (178 mL) in methanol was added. Stirring was turned off after 15 minutes and the clear mixture was filtered through a 0.45 μm nylon filter. Next, the filtrate was reduced in volume to 200 mL. Toluene (250 mL) was added and the mixture was again concentrated in vacuo to 200 mL. The resulting solution was transferred to a dropping funnel, washing the flask with 50 mL of toluene. This concentrate was added dropwise within 1 hour to 2,000 mL of hexane placed in a 4,000 mL flask, provided with a mechanical stirrer and an argon inlet. After completing addition of the concentrate the stirring was continued for another hour. The resulting suspension was filtered under reduced pressure of argon and washed with 400 mL of hexane. The precipitate was initially dried under a stream of argon, then under reduced pressure at room temperature for 12 hours and finally, under reduced pressure at 40° C. for 8 hours, to afford the sodium salt (53 g, purity 99.0% (HPLC)), identified as the amorphous sodium salt of montelukast.

1-20. (canceled)

21. tert-Butylammonium (R,E)-(1-{1-{3-[2-(7-chloroquinolin-2-yl)ethenyl]phenyl}-3-[2-(1-hydroxy-1-methylethyl)phenyl]-propylsulfanylmethyl}cyclopropyl)acetate.

22. The compound of claim 21 in a crystalline solid form.

23. A crystalline polymorph of the compound of claim 21 having at least one of the following: (a) an X-ray powder diffraction pattern substantially consistent with an X-ray powder diffraction pattern shown in FIG. 1; (b) a melting point (onset peak) of about 128.09° C.; or (c) X-ray diffraction pattern showing the peaks of relative intensity I/I₀ over 20% at the following 2θ angles:

| d (Å) | 2θ (°) | I/I₀ (%) |
|---|---|---|
| 3.30 | 26.74 | 24 |
| 8.89 | 9.94 | 75 |
| 14.26 | 6.21 | 28 |
| 14.67 | 6.03 | 29 |
| 16.22 | 5.46 | 24 |
| 17.03 | 5.20 | 100 |
| 17.86 | 4.96 | 49 |
| 19.87 | 4.46 | 87 |
| 20.43 | 4.34 | 96 |
| 21.01 | 4.22 | 33 |
| 21.69 | 4.09 | 24 |
| 22.67 | 3.92 | 62 |
| 23.86 | 3.73 | 85 |
| 25.61 | 3.48 | 59 |
| 26.15 | 3.40 | 77 |
| 26.74 | 3.33 | 27 |
| 27.96 | 3.19 | 31 |
| 28.69 | 3.11 | 33 |
| 30.35 | 2.94 | 33 |

24. A process for the preparation of tert-butylammonium (R,E)-(1-{1-{3-[2-(7-chloroquinolin-2-yl)ethenyl]phenyl}-3-[2-(1-hydroxy-1-methylethyl)phenyl]-propylsulfanylmethyl}cyclopropyl)acetate of the formula (4) 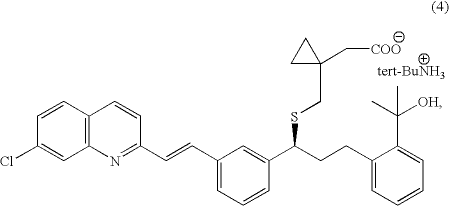
of claim 1 comprising the steps or:
(a) reacting a sulfonate derivative of (S)-1-{3-[2-(7-chloroquinolin-2-yl)ethylene]phenyl}-3-[2-(1-hydroxy-1-methylethyl)phenyl]propan-1-ol of the formula (2), 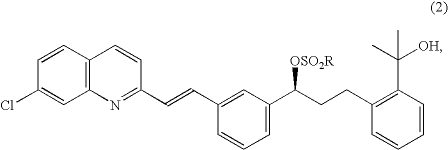
with a dianion of 1-(mercaptomethyl)-cyclopropaneacetic acid of the formula (3) 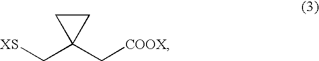
to yield a crude (R,E)-(1-{1-{3-[2-(7-chloroquinolin-2-yl)ethenyl]phenyl}-3-[2-(1-hydroxy-1-methylethyl)phenyl]propylsulfanylmethyl}cyclopropyl)acetic acid, wherein
R represents an alkyl or aryl moiety, and
X represents a sodium atom;
(b) reacting the crude (R,E)-(1-{1-{3-[2-(7-chloroquinolin-2-yl)ethenyl]phenyl}-3-[2-(1-hydroxy-1-methylethyl)phenyl]propylsulfanylmethyl}cyclopropyl)acetic acid obtained in step (a) with tert-butylamine to obtain tert-butylammonium (R,E)-(1-{1-{3-[2-(7-chloroquinolin-2-yl)ethenyl]phenyl}-3-[2-(1-hydroxy-1-methylethyl)phenyl]-propylsulfanylmethyl}cyclopropyl)acetate;
(c) isolating the tert-butylammonium (R,E)-(1-{1-{3-[2-(7-chloroquinolin-2-yl)ethenyl]phenyl}-3-[2-(1-hydroxy-1-methylethyl)phenyl]-propylsulfanylmethyl}cyclopropyl)acetate obtained in step (b); and
(d) optionally, recrystallizing the tert-butylammonium (R,E)-(1-{1-{3-[2-(7-chloroquinolin-2-yl)ethenyl]phenyl}-3-[2-(1-hydroxy-1-methylethyl)phenyl]-propylsulfanylmethyl}cyclopropyl)acetate obtained in step (c).

25. The process of claim 24, wherein, in step (a), the sulfonate derivative of (s)-1-{3-[2-(7-chloroquinolin-2-yl)ethylene]phenyl}-3-[2-(1-hydroxy-1-methylethyl)phenyl]propan-1-ol of the formula (2), 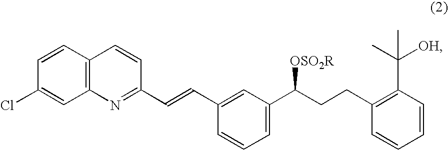
is (S)-1-{3-[2-(7-chloroquinolin-2-yl)ethylene]phenyl}-3-[2-(1-hydroxy-1-methylethyl)phenyl]-propan-1-ol methanesulfonate.

26. The process of claim 24, wherein the dianion of 1-(mercaptomethyl)-cyclopropaneacetic acid of the formula (3) used in step (a) is generated from the disodium salt of 1-(mercaptomethyl)-cyclopropaneacetic acid.

27. The process of claim 24, wherein the chemical purity of the tert-butylammonium (R,E)-(1-{1-{3-[2-(7-chloroquinolin-2-yl)ethenyl]phenyl}-3-[2-(1-hydroxy-1-methylethyl)phenyl]-propylsulfanylmethyl}cyclopropyl)acetate obtained in step (d) is greater than 99.0% (HPLC).

28. A pharmaceutical composition comprising a therapeutically effective amount of the compound of claim 21 and one or more pharmaceutically acceptable carriers and/or excipients.

29. A process for the preparation of (R,E)-(1-{1-{3-[2-(7-chloroquinolin-2-yl)ethenyl]phenyl}-3-[2-(1-hydroxy-1-methylethyl)phenyl]propylsulfanylmethyl}cyclopropyl)acetic acid of Formula (1) 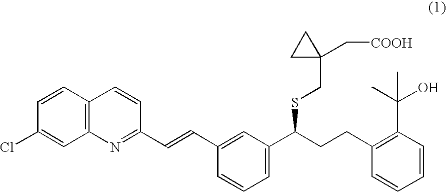
and/or pharmaceutically acceptable salts thereof, comprising the steps of:
(a) reacting a sulfonate derivative of (S)-1-{3-[2-(7-chloroquinolin-2-yl)ethylene]phenyl}-3-[2-(1-hydroxy-1-methylethyl)phenyl]propan-1-ol of the formula (2), 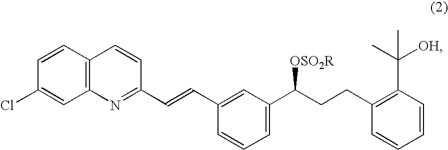
with a dianion of 1-(mercaptomethyl)-cyclopropaneacetic acid of the formula (3) 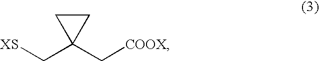
to yield a crude (R,E)-(1-{1-{3-[2-(7-chloroquinolin-2-yl)ethenyl]phenyl}-3-[2-(1-hydroxy-1-methylethyl)phenyl]propylsulfanylmethyl}cyclopropyl)acetic acid, wherein
R represents an alkyl or aryl moiety, and
X represents a sodium atom;
(b) reacting the crude (R,E)-(1-{1-{3-[2-(7-chloroquinolin-2-yl)ethenyl]phenyl}-3-[2-(1-hydroxy-1-methylethyl)phenyl]propylsulfanylmethyl}cyclopropyl)acetic acid obtained in step (a) with tert-butylamine to obtain a tert-butylamine salt of montelukast;
(c) isolating the tert-butylamine salt of montelukast from the reaction mixture;
(d) optionally, recrystallizing the tert-butylamine salt of montelukast;
(e) converting the tert-butylamine salt of montelukast to a free (R,E)-(1-{1-{3-[2-(7-chloroquinolin-2-yl)ethenyl]-phenyl}-3-[2-(1-hydroxy-1-methylethyl)phenyl]-propylsulfanyl-methyl}cyclopropyl)acetic acid; and
(f) optionally, converting the free (R,E)-(1-{1-{3-[2-(7-chloroquinolin-2-yl)ethenyl]-phenyl}-3-[2-(1-hydroxy-1-methylethyl)phenyl]-propylsulfanyl-methyl}cyclopropyl)acetic acid obtained in step (e) to a pharmaceutically-acceptable salt of montelukast.

30. The process of claim 29, wherein in step (a), the sulfonate derivative of (s)-1-{3-[2-(7-chloroquinolin-2-yl)ethylene]phenyl}-3-[2-(1-hydroxy-1-methylethyl)phenyl]propan-1-ol of the formula (2), 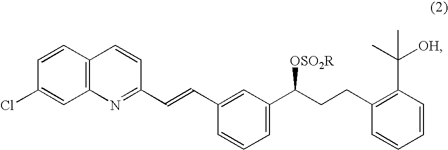
is (S)-1-{3-[2-(7-chloroquinolin-2-yl)ethylene]phenyl}-3-[2-(1-hydroxy-1-methylethyl)phenyl]-propan-1-ol methanesulfonate.

31. The process of claim 29, wherein the dianion of 1-(mercaptomethyl)-cyclopropaneacetic acid of the formula (3) used in step (a) is generated from the disodium salt of 1-(mercaptomethyl)-cyclopropaneacetic acid.

32. The process of claim 29, wherein the tert-butylamine salt of montelukast is isolated in step (c) by recrystallization.

33. The process of claim 32, wherein the tert-butylamine salt of montelukast is isolated in step (c) by recrystallization from toluene.

34. The process of claim 29, wherein the chemical purity of the tert-butylamine salt of montelukast obtained in step (d) is greater than 98.0% (HPLC).

35. The process of claim 29, wherein the chemical purity of the tert-butylamine salt of montelukast obtained in step (d) is greater than 99.0% (HPLC).

36. The process of claim 29, wherein the free (R,E)-(1-{1-{3-[2-(7-chloroquinolin-2-yl)ethenyl]-phenyl}-3-[2-(1-hydroxy-1-methylethyl)phenyl]-propylsulfanyl-methyl}cyclopropyl)acetic acid is obtained in step (e) by treating the tert-butylamine salt of montelukast with an aqueous solution of an organic mono- or dicarboxylic acid, or with a buffer solution.

37. The process of claim 29, wherein the chemical purity of the free (R,E)-(1-{1-{3-[2-(7-chloroquinolin-2-yl)ethenyl]phenyl}-3-[2-(1-hydroxy-1-methylethyl)phenyl]propylsulfanylmethyl}-cyclopropyl)acetic acid obtained in step (e) is greater than 99.0% (HPLC).

38. The process of claim 29, wherein the chemical purity of the free (R,E)-(1-{1-{3-[2-(7-chloroquinolin-2-yl)ethenyl]phenyl}-3-[2-(1-hydroxy-1-methylethyl)phenyl]propylsulfanylmethyl}-cyclopropyl)acetic acid obtained in step (e) is greater than 99.5% (HPLC)-.

39. The process of claim 29, wherein the free (R,E)-(1-{1-{3-[2-(7-chloroquinolin-2-yl)ethenyl]phenyl}-3-[2-(1-hydroxy-1-methylethyl)phenyl]propylsulfanylmethyl}cyclopropyl)acetic acid is obtained in step (e) in a crystalline form.

40. A crystalline polymorph of (R,E)-(1-{1-{3-[2-(7-Chloroquinolin-2-yl)ethenyl]phenyl}-3-[2-(1-hydroxy-1-methylethyl)phenyl]-propylsulfanylmethyl}cyclopropyl)acetic acid, said polymorph having at least one of the following: (a) an X-ray powder diffraction pattern substantially similar to an X-ray powder diffraction pattern shown in FIG. 2; (b) a melting point (onset peak) of about 153.20° C.; (c) a melting point (extrapolated peak) of about 155.61° C.; (d) an enthalpy of fusion of about −108.66 J/g; or (e) an X-ray powder diffraction pattern showing the peaks of relative intensity I/I₀ over 20% at the following 2θ angles of approximately:

| d (Å) | 2θ (°) | I/I₀ (%) |
|---|---|---|
| 9.92 | 8.91 | 58 |
| 15.40 | 5.75 | 100 |
| 17.84 | 4.97 | 21 |
| 18.22 | 4.87 | 27 |
| 20.32 | 4.37 | 57 |
| 20.72 | 4.28 | 28 |
| 21.11 | 4.20 | 25 |
| 21.50 | 4.13 | 20 |
| 23.13 | 3.84 | 22 |
| 24.51 | 3.63 | 65 |
| 26.24 | 3.39 | 32 |
| 27.81 | 3.21 | 26 |
